(19) United States
(12) Patent Application Publication
(10) Pub. No.: US 2023/0042491 A1
(43) Pub. Date: Feb. 9, 2023

(54) EVALUATION SYSTEM AND EVALUATION METHOD

(71) Applicant: NATIONAL RESEARCH AND DEVELOPMENT AGENCY NATIONAL, Ibaraki (JP)

(72) Inventor: Makoto MITSUMORI, Ibaraki (JP)

(21) Appl. No.: 17/787,849

(22) PCT Filed: Nov. 27, 2020
(86) PCT No.: PCT/JP2020/044171

§ 371 Date: Jun. 21, 2022

(30) Foreign Application Priority Data
Dec. 23, 2019 (JP) 2019-232023

(51) Int. Cl. A01J 5/01; A01K 29/00; A61B 5/00

(52) U.S. Cl. CPC A01J 5/01; A01K 29/005; A61B 5/4288; A61B 5/7278; A61B 5/4866; A61B 2503/40

(57) ABSTRACT

The evaluation system for evaluating a status of rumen fermentation in a ruminant includes an evaluation device that has an estimation unit for estimating a status of rumen fermentation in a ruminant based on a milk production record which indicates a milk yield and a milk component of milk produced by the ruminant. According to the evaluation system, it is possible to easily evaluate the status of rumen fermentation in the ruminant.

### TECHNICAL FIELD

The present invention relates to an evaluation system and an evaluation method.

### BACKGROUND ART

Livestock which is a ruminant such as a cow is herbivorous, and digests feed derived from plants to produce milk and meat. A ruminant digests plant feed with four stomachs. Of the four stomachs, the first stomach called a rumen has a large capacity of approximately 100 liters and is internally symbiotic with a wide variety of rumen microorganisms. Plant feed ingested by the ruminant is first degraded by fermentation (rumen fermentation) by the rumen microorganisms, becomes sources of energy and proteins for the ruminant, and thus supports growth of the ruminant and production of milk and meat.

It is known that acidification of rumen fluid (ruminal acidosis) is caused by feeding a large amount of cereal feed to a ruminant in anticipation of an increase in milk yield, improvement in milk quality, and the like. Ruminal acidosis is a cause of a so-called production disease such as dyspepsia, a decrease in feed intake and in milk yield, and a decrease in conception rate, which reduces productivity of ruminants. Therefore, it is important to properly feed so that rumen fermentation functions normally in order to maintain the productivity of ruminants. Under the circumstances, it is demanded to manage feeding while taking into consideration a status of rumen fluid, which reflects a status of rumen fermentation.

Conventionally, rumen fermentation has been evaluated by measuring a feed intake (dry matter intake: DMI) and pH of rumen fluid (ruminal pH) of livestock. Non-Patent Literature 1 proposes that ruminal pH, a short-chain fatty acid concentration in rumen fluid, a DMI, a body weight of a cow, and the like are measured, and a theoretical turnover rate (TTOR) of a rumen liquid fraction calculated from these measured values can be used as a new indicator for evaluating rumen fermentation.

### CITATION LIST

### Non-Patent Literature

### [Non-patent Literature 1]

Mitsumori et. al., Animal Science Journal, http://doi.org/10.1111/asj.13305, 2019/10/24, [online]

### SUMMARY OF INVENTION

### Technical Problem

Conventionally, a DMI has been determined by subtracting residual feed, which has not been taken, from supplied feed. However, it is extremely difficult for an ordinary farmer to weigh supplied feed and residual feed. Moreover, ruminal pH is measured by measuring, with a pH meter, rumen fluid collected with a gastric catheter or the like, or by using a pH sensor provided to the rumen. However, it is not easy for an ordinary farmer to collect rumen fluid or provide a pH sensor. Under the circumstances, if it is possible to evaluate a status of rumen fermentation easily and practically for ordinary farmers, such a technique would be very beneficial.

An object of an aspect of the present invention is to provide an evaluation system that can easily evaluate a status of rumen fermentation.

### Solution to Problem

In order to attain the object, the present invention includes the following aspects:

an evaluation system for evaluating a status of rumen fermentation in a ruminant, the evaluation system including an evaluation device that has an estimation unit, the estimation unit estimating a status of rumen fermentation in an evaluation subject ruminant based on a milk production record which indicates a milk yield and a milk component of milk produced by the evaluation subject ruminant; and

an evaluation method for evaluating a status of rumen fermentation in a ruminant, the method including an estimation step of estimating a status of rumen fermentation in a ruminant based on a milk production record which indicates a milk yield and a milk component of milk produced by the ruminant.

### Advantageous Effects of Invention

According to an aspect of the present invention, it is possible to easily evaluate a status of rumen fermentation without weighing supplied feed and residual feed and without measuring pH of rumen fluid.

### BRIEF DESCRIPTION OF DRAWINGS

FIG. 1 is a schematic diagram illustrating a correlation between indicators used in an evaluation system in accordance with an embodiment of the present invention.

FIG. 2 is a block diagram schematically illustrating the evaluation system in accordance with an embodiment of the present invention.

FIG. 3 is a graph showing a correlation between (i) a theoretical turnover rate (TTOR) of a rumen liquid fraction calculated from a short-chain fatty acid concentration in a rumen and a methane yield and (ii) an estimated value of TTOR estimated from a milk production record.

FIG. 4 is a graph showing a correlation between (i) an estimated value of TTOR and a milk production record and (ii) a measured value of ruminal pH.

FIG. 5 is a graph showing a correlation between (i) an estimated value of TTOR and a milk production record and (ii) a measured value of ruminal pH.

FIG. 6 is a graph showing a correlation between (i) an estimated value of TTOR and a dry matter intake and (ii) the estimated value of TTOR and a milk yield.

FIG. 7 is a graph showing a correlation between (i) an estimated value of TTOR and a dry matter intake and (ii) the estimated value of TTOR and a milk yield.

FIG. 8 is a graph showing a correlation between (i) an estimated value of TTOR and a dry matter intake and (ii) the estimated value of TTOR and a milk yield.

### DESCRIPTION OF EMBODIMENTS

The following will describe details of an embodiment of the present invention.

[Evaluation System]

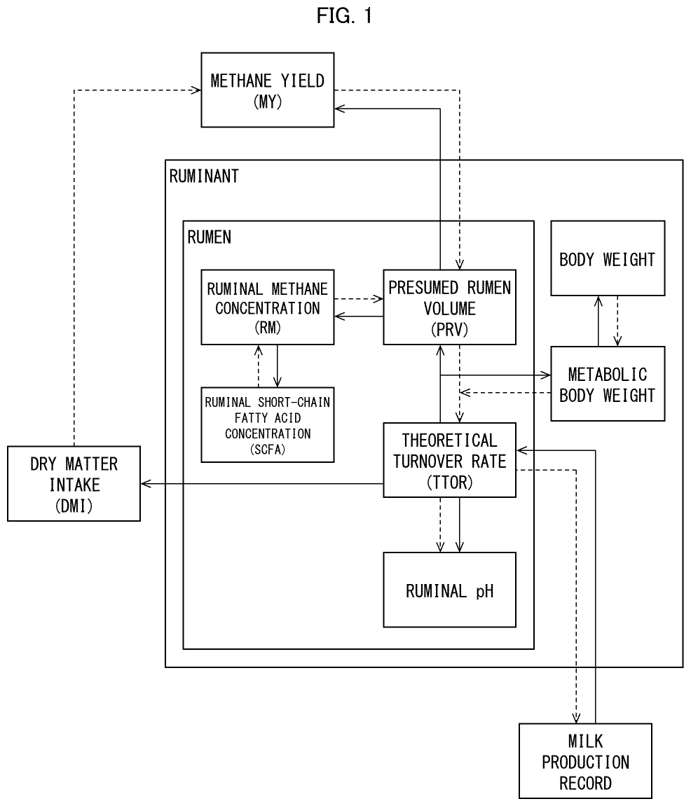

The evaluation system in accordance with an embodiment of the present invention is an evaluation system for evaluating a status of rumen fermentation in a ruminant. The evaluation system evaluates a status of rumen fermentation by rumen microorganisms in the first stomach, called a rumen, of a ruminant. In the evaluation system, a status of rumen fermentation in a ruminant is evaluated based on various indicators on energy metabolism of the ruminant (see FIG. 1).

[Correlation Between Indicators Representing Status of Rumen Fermentation]

FIG. 1 is a schematic diagram illustrating a correlation between indicators used in an evaluation system in accordance with an embodiment of the present invention. As illustrated in FIG. 1, it is disclosed in Non-Patent Literature 1 that various indicators on energy metabolism of a ruminant are related to each other. Here, Non-Patent Literature 1 is entirely incorporated as reference into the present specification. Hereinafter, Non-Patent Literature 1 is referred to as Reference Document 1. Reference Document 1 indicates that there is a close association between (i) a theoretical turnover rate (TTOR) of a rumen liquid fraction calculated from ruminal pH obtained from a pH monitor anchored in the rumen, a ruminal short-chain fatty acid concentration (SCFA concentration: short-chain fatty acid concentration in a rumen), a dry matter intake (DMI), and a body weight of a cow, and (ii) the DMI, a milk yield, and the ruminal pH. In FIG. 1, the dashed line arrows indicate a correlation between indicators shown in Reference Document 1, and the solid line arrows indicate a correlation between indicators revealed by the present invention.

A TTOR is an indicator representing a status of rumen fermentation. Here, the status of rumen fermentation is represented by various indicators relating to fermentation of feed in the rumen. In addition to the TTOR, examples of the indicators representing a status of rumen fermentation include ruminal pH, a DMI, an SCFA concentration, an SCFA amount, a methane concentration in a rumen liquid fraction, a methane yield, and the like.

In FIG. 1, as indicated by the dashed line arrows, the TTOR can be calculated from a methane yield emitted by a ruminant and an SCFA concentration. Specifically, a presumed rumen volume (PRV) is calculated from the methane yield and a methane concentration in a rumen liquid fraction calculated from the SCFA concentration, and the TTOR is calculated using this PRV and a metabolic body weight (MBW) of the ruminant.

A methane yield can be calculated by a known method based on a DMI. The DMI can be obtained by weighing supplied feed and residual feed and subtracting the residual feed from the supplied feed. A methane concentration in a rumen liquid fraction can be calculated from a flow of metabolic hydrogen in rumen fermentation based on an SCFA concentration measured in rumen fluid taken from the rumen. A presumed rumen volume represents an estimated value of a total volume of a liquid phase portion in a rumen. A metabolic body weight is a value calculated by 0.75th power of a body weight of a ruminant.

Ruminal pH is an important indicator for evaluating ruminal acidosis. Conventionally, ruminal pH has been measured by measuring, with a pH meter, rumen fluid taken with a gastric catheter or the like, or by using a pH sensor provided to the rumen. Reference Document 1 indicates that, without using such a measuring method, an indicator (such as ruminal pH) representing a status of rumen fermentation can be estimated from a TTOR calculated from a methane yield and an SCFA concentration.

As indicated in Reference Document 1, it is very advantageous if ruminal pH and the like can be estimated from the TTOR, because it is not necessary to collect rumen fluid every time a status of rumen fermentation is evaluated or to provide a pH sensor in the rumen. However, in particular for an ordinary farmer, it is not easy to calculate a methane yield and an SCFA concentration, and consequently it is not easy to calculate a TTOR. Therefore, it is further advantageous if a status of rumen fermentation can be more easily estimated without calculating a TTOR.

The inventors of the present invention have found, for the first time, that it is possible to estimate a status of rumen fermentation based on a milk production record which indicates a milk yield and a milk component of milk produced by a ruminant, and thus have accomplished the present invention. Milk produced by a ruminant is obtained by milking from the ruminant, and can be easily obtained by an ordinary farmer because it is necessary to carry out milking several times every day in a lactation period. A milk yield and a milk component of milk produced by a ruminant are also routinely measured by an ordinary farmer in order to evaluate the milk produced.

Therefore, as a measure for easily evaluating a status of rumen fermentation, the present invention is extremely advantageous because it is possible to estimate a status of rumen fermentation based on a milk production record of milk produced by a ruminant, without using indicators such as a methane yield and an SCFA concentration.

[Ruminant]

A subject of evaluation by the evaluation system is ruminal livestock. A ruminant is an animal that has four stomachs for digesting feed which is mainly constituted by plant-derived components, and that carries out rumination. Ruminal livestock, which is to be evaluated by the evaluation system, includes a cow, a goat, a sheep, and the like, and is typical a cow. In the evaluation system, a status of rumen fermentation in a ruminant is evaluated based on milk produced by the ruminant. The evaluation system is therefore particularly suitable for evaluating a dairy cow from which milk is routinely collected and for which a yield and components of the milk are monitored.

[Milk Production Record]

In the evaluation system, a status of rumen fermentation is evaluated on the basis of a milk production record which indicates a milk yield and a milk component of milk produced by an evaluation subject ruminant. The milk component can include at least one of a milk fat proportion, a milk protein proportion, a solid non fat proportion, a milk sugar proportion, an ammonia nitrogen proportion, and a component ratio such as a milk protein/milk fat ratio. Those indicators on milk production are routinely measured to manage milk production at a dairy farm.

A milk yield means a daily weight of milk produced. A milk fat proportion means a weight ratio of fat to a milk yield. A milk protein proportion means a weight ratio of protein to a milk yield. A solid non fat proportion means a weight ratio of components excluding water and fat to a milk yield. A milk sugar proportion means a weight ratio of milk sugar to a milk yield. An ammonia nitrogen proportion means a ratio of nitrogen contained in urea to a milk yield. A milk protein/milk fat ratio means a weight ratio between protein and fat to a milk yield. Methods of measuring a milk yield and a milk component are not particularly limited, and the milk yield and the milk component can be measured by conventionally known methods.

The milk production record can include at least one value calculated from a combination of two or more of the foregoing values of the milk component. For example, a status of rumen fermentation can be evaluated by using, as the milk production record, a value calculated from a combination of the milk protein proportion and the milk protein/milk fat ratio.

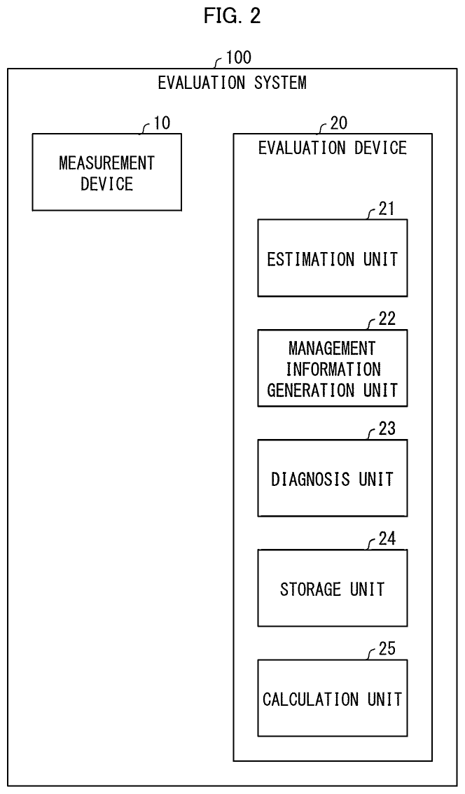

The following description will discuss a configuration of the evaluation system with reference to FIG. 2. FIG. 2 is a block diagram schematically illustrating the evaluation system in accordance with an embodiment of the present invention. As illustrated in FIG. 2, an evaluation system 100 includes an evaluation device 20. The evaluation device 20 has an estimation unit 21. The evaluation device 20 can further include a management information generation unit 22, a diagnosis unit 23, a storage unit 24, and a calculation unit 25. The evaluation system 100 can further include a measurement device 10.

[Measurement Device]

The measurement device 10 measures a milk yield and a milk component of milk produced by an evaluation subject ruminant. As the measurement device 10, a conventionally known device for measuring a milk yield and a milk component of milk collected from a ruminant can be used. The measurement device 10 is preferably an automatic analyzer for automatically analyzing collected milk. The measurement device 10 sends a result of the measured milk production record to the evaluation device 20.

Further, the measurement device 10 can have a function of measuring pH of rumen fluid collected from a ruminant, an SCFA concentration in the rumen fluid, and the like.

[Evaluation Device]

The evaluation device 20 evaluates a status of rumen fermentation in an evaluation subject ruminant. The evaluation device 20 evaluates a status of rumen fermentation based on a result of the milk production record which indicates a milk yield and a milk component and has been measured by the measurement device 10.

(Estimation Unit)

The estimation unit 21 estimates a status of rumen fermentation in an evaluation subject ruminant on the basis of a milk production record which indicates a milk yield and a milk component of milk produced by the evaluation subject ruminant. A status of rumen fermentation estimated by the estimation unit 21 is, for example, a TTOR, ruminal pH, and a DMI. The estimation unit 21 can also estimate, based on a TTOR estimated based on a milk production record, indicators such as an SCFA concentration, an SCFA amount, a methane concentration in a rumen liquid fraction, and a methane yield, which represent a status of rumen fermentation.

As described later, the estimation unit 21 estimates a status of rumen fermentation using a relational expression representing a correlation between indicators representing the status of rumen fermentation. Such a relational expression can be obtained in the evaluation system 100, or can be obtained externally and stored in the evaluation system 100. Therefore, for example, an ordinary farmer does not need to carry out measurement or calculation in order to obtain such a relational expression, and it is possible to estimate a status of rumen fermentation only by obtaining a relational expression from an external source and substituting a milk production record into the relational expression.

<Estimation of TTOR>

The estimation unit 21 estimates a TTOR based on, for example, a milk production record as indicated below. The estimation unit 21 estimates a TTOR from a measurement result in a milk production record of milk produced by an evaluation subject ruminant, based on a correlation between (i) a TTOR calculated based on a methane yield emitted by a ruminant and an SCFA concentration and (ii) a milk production record of milk produced by the ruminant.

The correlation between the calculated TTOR and the milk production record of milk produced by the ruminant, which is used by the estimation unit 21, is represented by a regression equation obtained by a regression analysis. The regression equation can be directly obtained from plots on a scatter diagram that has been prepared from (i) a TTOR calculated based on a methane yield emitted by a ruminant and an SCFA concentration and (ii) a milk production record of milk produced by the ruminant. For example, in the scatter diagram, a regression line is obtained by a least squares method, and a regression equation is obtained from the regression line thus obtained.

Then, the estimation unit 21 calculates a TTOR by substituting the value of the milk production record of milk produced by the evaluation subject ruminant into the obtained regression equation. Thus, the estimation unit 21 can calculate a TTOR only by substituting a value of a milk production record of milk produced by an evaluation subject ruminant into the foregoing regression equation. The regression equation used by the estimation unit 21 can be obtained in advance by the calculation unit 25 (described later), or can be obtained in advance from an external source.

<Estimation of Ruminal pH>

The estimation unit 21 can estimate pH in a rumen of an evaluation subject ruminant from a measurement result in a milk production record of the evaluation subject ruminant, based on a TTOR (i.e., a theoretical turnover rate of a rumen liquid fraction) which has been estimated from the milk production record as described above. The estimation unit 21 estimates ruminal pH from a measurement result in a milk production record of milk produced by an evaluation subject ruminant based on, for example, a correlation between an estimated TTOR and a measured value of ruminal pH.

The correlation between the estimated TTOR and the measured value of ruminal pH, which is used by the estimation unit 21, is represented by a regression equation obtained by a regression analysis. The regression equation can be directly obtained from plots on a scatter diagram that has been prepared from, for example, (i) an estimated TTOR and a measured value of a milk production record and (ii) a measured value of ruminal pH. For example, in the scatter diagram, a regression line is obtained by a least squares method, and a regression equation is obtained from the regression line thus obtained.

Then, the estimation unit 21 calculates ruminal pH by substituting the value of the milk production record of milk produced by the evaluation subject ruminant into the obtained regression equation. Thus, the estimation unit 21 can calculate ruminal pH only by substituting a value of a milk production record of milk produced by an evaluation subject ruminant into the foregoing regression equation. The regression equation used by the estimation unit 21 can be obtained in advance by the calculation unit 25 (described later), or can be obtained in advance from an external source.

<Estimation of DMI>

The estimation unit 21 can estimate a DMI of an evaluation subject ruminant from a measurement result in a milk production record of the evaluation subject ruminant, based on a TTOR which has been estimated from the milk production record as described above. The estimation unit 21 estimates a DMI from a measurement result in a milk production record of milk produced by an evaluation subject ruminant based on, for example, a correlation between an estimated TTOR and a measured value of DMI.

The correlation between the estimated TTOR and the measured value of DMI, which is used by the estimation unit 21, is represented by a regression equation obtained by a regression analysis. The regression equation can be directly obtained from plots on a scatter diagram that has been prepared from, for example, (i) an estimated TTOR and a value related to a measured value of a milk production record and (ii) the estimated TTOR and a value related to a measured value of DMI. For example, in the scatter diagram, a regression line is obtained by a least squares method, and a regression equation is obtained from the regression line thus obtained.

Then, the estimation unit 21 calculates a DMI by substituting the value of the milk production record of milk produced by the evaluation subject ruminant into the obtained regression equation. Thus, the estimation unit 21 can calculate a DMI only by substituting a value of a milk production record of milk produced by an evaluation subject ruminant into the foregoing regression equation. The regression equation used by the estimation unit 21 can be obtained in advance by the calculation unit 25 (described later), or can be obtained in advance from an external source.

The estimation unit 21 sends an indicator representing the estimated status of rumen fermentation to the management information generation unit 22, the diagnosis unit 23, the storage unit 24, and the calculation unit 25 (which will be described later).

(Management Information Generation Unit)

The management information generation unit 22 generates feeding management information pertaining to a ruminant based on a status of rumen fermentation in the ruminant, which has been estimated by the estimation unit 21. The feeding management information includes pieces of information on a feeding amount (such as DMI), a feeding frequency, a feeding time, a type of feed, components of feed, a component ratio of feed, water consumption, and the like. The management information generation unit 22 generates feeding management information by taking into consideration, in addition to a status of rumen fermentation in a ruminant, pieces of information such as a variety of ruminant, an age (month or year), a body weight, a parturition date, the number of days after parturition, estrus, pregnancy, a lactation period, a milking frequency, a dry period, as well as a temperature, humidity, a wind speed, a rainfall, and a location (address) of a feeding management place, and a management style.

The management information generation unit 22 generates, based on, for example, predetermined data in which a status of rumen fermentation is associated with feeding management information, feeding management information corresponding to a status of rumen fermentation estimated by the estimation unit 21 as feeding management information pertaining to an evaluation subject ruminant.

The management information generation unit 22 can send the generated feeding management information to the storage unit 24 or can display the generated feeding management information on a display unit (not illustrated) to notify the user.

(Diagnosis Unit)

The diagnosis unit 23 diagnoses at least one of a feeding management status and a production disease of a ruminant based on a status of rumen fermentation in the ruminant, which has been estimated by the estimation unit 21.

The production disease of a ruminant includes ruminal acidosis, dyspepsia, loose stool, a decrease in feed intake, an increase or decrease in milk component such as a decrease in milk fat proportion, a decrease in milk yield, development of laminitis, a decrease in conception rate, and the like. The diagnosis unit 23 diagnoses, based on, for example, predetermined data in which a status of rumen fermentation is associated with a morbidity probability of a production disease, a morbidity probability of a production disease corresponding to a status of rumen fermentation estimated by the estimation unit 21, as a morbidity probability of a production disease of an evaluation subject ruminant.

The feeding management status of a ruminant includes various items related to feeding management, such as a feed intake, a body weight, a body condition score, a rumen fill score, a fecal score, a claudication score (locomotion score), blood components (such as glucose, free fatty acid (NEFA), β-hydroxybutyric acid (BHBA), calcium, total protein, albumin, aspartate aminotransferase (AST), gamma-glutamyl transpeptidase (GGT), ammonia nitrogen, glucose, triglyceride, total cholesterol (T-Cho), insulin, and luteinizing hormone), urinalysis results (such as uric acid, pH, and creatinine), a milk production record representing a milk yield and a milk component of milk, and a milking frequency per day. The diagnosis unit 23 diagnoses whether or not a feeding management status corresponding to a status of rumen fermentation estimated by the estimation unit 21 is proper, based on, for example, predetermined data in which a status of rumen fermentation is associated with a proper feeding management status.

The diagnosis unit 23 can send information pertaining to a diagnosis result of a feeding management status and a production disease to the storage unit 24, or can display the information on a display unit (not illustrated) to notify the user.

(Storage Unit)

The storage unit 24 stores information indicating a correlation between a milk production record and a status of rumen fermentation in a ruminant. The storage unit 24 stores, for example, a regression equation representing a correlation between a TTOR and a milk production record, a regression equation representing a correlation between a TTOR and ruminal pH, a regression equation representing a correlation between a TTOR and a DMI, and the like. The storage unit 24 can store an indicator representing a status of rumen fermentation estimated by the estimation unit 21. Further, the storage unit 24 can store feeding management information generated by the management information generation unit 22 and information pertaining to a production disease diagnosed by the diagnosis unit 23. The storage unit can be, for example, a conventionally known computer memory.

(Calculation Unit)

The calculation unit 25 calculates a relational expression representing a correlation between a milk production record and a status of rumen fermentation in a ruminant. The calculation unit 25 calculates, for example, a regression equation representing a correlation between a TTOR and a milk production record, a regression equation representing a correlation between a TTOR and ruminal pH, and a regression equation representing a correlation between a TTOR and a DMI.

For example, the calculation unit 25 first calculates a TTOR from measured values of a methane yield emitted by a ruminant and an SCFA concentration in a sample of a population. Then, the calculation unit 25 calculates a regression equation representing a correlation between the TTOR and a milk production record by regression analysis of a correlation between the calculated TTOR and the measured value of the milk production record in the sample of the population. The calculation unit 25 can calculate a methane yield based on a DMI by a known method. Further, the calculation unit 25 can calculate a methane concentration in a rumen liquid fraction from a flow of metabolic hydrogen in rumen fermentation based on an SCFA concentration measured in rumen fluid collected from a rumen.

The population can be a cluster of ruminants reared at a particular farm, can be a cluster of ruminants reared under a particular rearing condition, or can be a cluster of ruminal livestock including two or more clusters of ruminants. The population is preferably a cluster of ruminants reared under a rearing condition similar to that for an evaluation subject ruminant, and more preferably a cluster of ruminants reared at the same farm as an evaluation subject ruminant. By using a relational expression calculated from such a population, it is possible to more accurately estimate a status of rumen fermentation. The population can be a cluster similar to an evaluation subject ruminant in terms of individual conditions such as a variety, an age, and the number of deliveries, and feed conditions such as a type, a composition, and a supplied amount of feed. A status of rumen fermentation in a ruminant individual can be estimated by using a relational expression obtained by calculating, as a population, a group of pieces of data obtained at different times for the ruminant individual. Thus, it is possible to analyze each individual.

[Evaluation Method]

The evaluation method in accordance with an embodiment of the present invention is an evaluation method for evaluating a status of rumen fermentation in a ruminant. The evaluation method includes an estimation step of estimating a status of rumen fermentation in a ruminant based on a milk production record which indicates a milk yield and a milk component of milk produced by the ruminant. In other words, the evaluation method is an aspect of an evaluation process in the foregoing evaluation system in accordance with an embodiment of the present invention. Therefore, the descriptions of the foregoing evaluation system in accordance with an embodiment of the present invention apply to details of the evaluation method.

[Software Implementation Example]

The evaluation device in accordance with an aspect of the present invention can be realized by a computer. In such a case, the present invention encompasses (i) a control program of the evaluation device which causes the computer to serve as the units (software elements) included in the evaluation device for realizing the evaluation device and (ii) a computer-readable storage medium storing the control program.

A control block (particularly, the estimation unit 21 and the calculation unit 25) of the evaluation device 20 can be realized by a logic circuit (hardware) provided in an integrated circuit (IC chip) or the like or can be alternatively realized by software.

In the latter case, the evaluation device 20 includes a computer which executes instructions of a program that is software realizing the foregoing functions. The computer includes, for example, at least one processor and a computer-readable storage medium storing the program. In a case where the processor in the computer reads out the program from the storage medium and executes the program, the object of the present invention is achieved. Examples of the processor encompass a central processing unit (CPU). Examples of the storage medium encompass a “non-transitory tangible medium” such as a read only memory (ROM), a tape, a disk, a card, a semiconductor memory, and a programmable logic circuit. The computer may further include a random access memory (RAM) or the like in which the program is loaded. Further, the program may be made available to the computer via any transmission medium (such as a communication network and a broadcast wave) which allows the program to be transmitted. Note that an aspect of the present invention can also be achieved in the form of a computer data signal in which the program is embodied via electronic transmission and which is embedded in a carrier wave.

The present invention can also be expressed as follows:

1) An evaluation system for evaluating a status of rumen fermentation in a ruminant, the evaluation system including an evaluation device that has an estimation unit, the estimation unit estimating a status of rumen fermentation in an evaluation subject ruminant based on a milk production record which indicates a milk yield and a milk component of milk produced by the evaluation subject ruminant.
2) The evaluation system described in 1), in which: the estimation unit estimates a theoretical turnover rate of a rumen liquid fraction based on the milk production record of milk produced by the evaluation subject ruminant, the theoretical turnover rate indicating the status of rumen fermentation in the evaluation subject ruminant.
3) The evaluation system described in 2), in which: the estimation unit estimates the theoretical turnover rate of the rumen liquid fraction of the evaluation subject ruminant from a measurement result in the milk production record of the evaluation subject ruminant based on a correlation between (i) a theoretical turnover rate of a rumen liquid fraction which has been calculated based on a methane yield released by a ruminant and a ruminal short-chain fatty acid concentration in the ruminant and (ii) the milk production record.
4) The evaluation system described in 2) or 3), in which: the estimation unit estimates ruminal pH of the evaluation subject ruminant from a measurement result in the milk production record of the evaluation subject ruminant based on an estimated ruminal theoretical turnover rate which has been estimated from the milk production record.
5) The evaluation system described in 2) or 3), in which: the estimation unit estimates a dry matter intake from a measurement result in the milk production record of the evaluation subject ruminant based on an estimated ruminal theoretical turnover rate which has been estimated from the milk production record.
6) The evaluation system described in any of 1) through 5), in which: the evaluation device further includes a management information generation unit that generates feeding management information pertaining to a ruminant based on a status of rumen fermentation in the ruminant, which has been estimated by the estimation unit.
7) The evaluation system described in any of 1) through 6), in which: the evaluation device further includes a diagnosis unit that diagnoses at least one of a feeding management status and a production disease of a ruminant based on a status of rumen fermentation in the ruminant, which has been estimated by the estimation unit.
8) The evaluation system described in any of 1) through 7), in which: the evaluation device further includes a storage unit that stores information indicating a correlation between the milk production record and the status of rumen fermentation in the ruminant.
9) The evaluation system described in any of 1) through 8), in which: the evaluation device further includes a calculation unit that calculates a relational expression indicating a correlation between the milk production record and the status of rumen fermentation in the ruminant.
10) The evaluation system described in any of 1) through 9), in which: the milk component includes at least one of a milk fat proportion, a milk protein proportion, a solid non fat proportion, a milk sugar proportion, an ammonia nitrogen proportion, and a milk protein/milk fat ratio.
11) The evaluation system described in 10), in which: the milk production record includes at least one value calculated from a combination of two or more values of the milk component.
12) An evaluation method for evaluating a status of rumen fermentation in a ruminant, the method including: an estimation step of estimating a status of rumen fermentation in a ruminant based on a milk production record which indicates a milk yield and a milk component of milk produced by the ruminant.

The present invention is not limited to the embodiments, but can be altered by a skilled person in the art within the scope of the claims. The present invention also encompasses, in its technical scope, any embodiment derived by combining technical means disclosed in differing embodiments.

### EXAMPLES

The following description will discuss an Example of the present invention.

[1. Calculation of TTOR Based on Measured Values of DMI and Rumen Fluid Component]

The experiments of 1-1. through 1-6. are based on the descriptions of Reference Document 1. The pieces of data measured in Reference Document 1 were obtained at different times for each individual cow.

(1-1. Animal Handling and Sampling)

Eleven Holstein dairy cows (multiparous) reared at research institutes in Chiba prefecture, Ibaraki prefecture, Ishikawa prefecture, Kanagawa prefecture and Toyama prefecture, Japan were used in this experiment. Those dairy cows were reared in cowhouses with tie stalls at the institutes and fed a commercially available dry blended ration twice a day (at 09:00 and 16:00) at a concentration that meets 120% of energy requirements according to the Japan Feeding Standard (NARO, 2006) during 3 weeks prior to parturition.

After that, in a lactation period, the dairy cows were fed a ration including timothy hay twice a day (at 09:00 and 16:00) at a concentration that meets 100% of energy requirements according to the Japan Feeding Standard (NARO, 2006). As a difference in amount between supplied feed and residual feed, a DMI was measured daily for each cow throughout the experimental period.

Ruminal pH was measured for each dairy cow by using a radio transmission pH sensor attached to the stomach of that dairy cow during the testing period, i.e., 3 weeks prepartum to 12 weeks postpartum. Ruminal pH values were continuously recorded every 10 minutes throughout the measurement. The pH measured at 13:00 was taken as a representative value of the daily ruminal pH.

This experiment was carried out in accordance with the Japanese Standards Relating to the Care and Management of Experimental Animals, and the experimental procedures used were under a unified protocol. The protocol was approved by the Animal Care Committee of the Institute of Livestock and Grassland Science, NARO, Japan.

A milk yield of each dairy cow was measured daily, and milk components were analyzed weekly. Rumen fluid samples were taken via a stomach tube from the dairy cow at 4 hours after morning feeding. The sampling of rumen fluid was carried out at 3 weeks before parturition and at 4, 8, and 12 weeks after parturition. Rumen fluid was strained through four layers of cheesecloth, and the fluid was stored at −20° C. until further analysis.

(1-2. Component Analysis)

Sampling of blood from eleven dairy cows was carried out at 3 weeks before parturition and at 4, 8, and 12 weeks after parturition. Blood samples were each collected from the coccygeal vein by a suction tube containing an anticoagulant, and analyzed as described in Hasunuma et al., 2016.

Plasma concentrations of total protein, albumin, aspartate aminotransferase (AST), gamma-glutamyl transpeptidase (GGT), ammonia nitrogen, glucose, triglyceride, total cholesterol (T-Cho), and non-esterified fatty acid were analyzed using an automatic analyzer (Model 7020; available from Hitachi, Ltd). A concentration of organic acid in the rumen fluid was measured by high-performance liquid chromatography (available from Alliance HPLC system; Waters, Milford). Concentrations of milk fat, milk protein, solid non fat, somatic cells, and ammonia nitrogen were determined using an automatic analyzer at each experimental institute.

(1-3. Statistical Analysis)

A statistical analysis was carried out by a two-way analysis of variance (ANOVA) followed by a Tukey's multiple-comparison post hoc test, and a significant difference was determined by a method of least significant difference at 5% (P<0.05) using Excel 2011 software (Microsoft) with add-in software Statcel3 (OMS Publishing). A simple regression analysis was carried out.

(1-4. Method for Calculating TTOR)

A correlation between pieces of data used in a theoretical analysis for a turnover rate of rumen fluid is illustrated in FIG. 1. A methane yield (MY) was estimated from a DMI using the following equation (1-1).

MY(mol/day)=[19.14×DMI(kg/day)+2.54]/16.042 (1-1):

A methane concentration in a rumen liquid fraction (RM) was calculated from a flow of metabolic hydrogen in rumen fermentation. That is, the methane concentration in a rumen liquid fraction (RM) was calculated based on metabolic hydrogen that was used in rumen fermentation (HU) and metabolic hydrogen that was produced (HP).

Here, the HP which is a fermentation intermediate and hydrogen in short-chain fatty acid in the rumen liquid fraction (HUS) were estimated from amounts and molar proportions of acetic acid (C2), propionic acid (C3) and butyric acid (C4) in rumen fluid, using the following equations (1-2) and (1-3). The short-chain fatty acid is a main energy source for performance of a host and a ruminant.

HP(mM)=2×C2+C3+4×C4 (1-2):

HUS(mM)=2×C3+2×C4 (1-3):

In a case where a sum of metabolic hydrogen used to produce short-chain fatty acid in rumen fermentation (HUS) and metabolic hydrogen used to purify methane in rumen fermentation (HUM) is defined as metabolic hydrogen used in rumen fermentation (HU), a recovery rate of metabolic hydrogen produced by rumen fermentation (HP) to metabolic hydrogen used in rumen fermentation (HU) is estimated to be 0.9 (Demeyer, 1991). Therefore, it is possible to calculate the metabolic hydrogen used in rumen fermentation (HU) from the following equation (1-4).

HU=0.9×HP=HUS+HUM (1-4):

From this, metabolic hydrogen used in the rumen (HU) was calculated from the following equation (1-5) (Goel, Makkar and Becker (2009)).

HU=HUS+HUM=(2×C3+2×C4)+(4×methane) (1-5):

From these, an equation for calculating a methane concentration in a rumen liquid fraction (RM) is expressed as the following equation (1-6).

RM(mM)=(HU−HUS)/4=[(0.9×HP)−HUS]/4 (1-6):

The inventors of the present invention calculated a presumed rumen volume (PRV) from the following equation (1-7).

PRV(L/day)=MY(mol/day)/[RM(mM)/1000] (1-7):

A short-chain fatty acid yield was calculated from the following equation (1-8) using a ruminal concentration of short-chain fatty acid (SCFA) and a presumed rumen volume (PRV). The inventors of the present invention calculated a short-chain fatty acid yield using the MY and RM concentration which had been estimated from the DMI and ruminal concentration of short-chain fatty acid.

Short-chain fatty acid yield(mol/day)=SCFA (mM)×PRV (1-8):

The TTOR was calculated using a metabolic body weight (MBW), which is represented by (body weight)^{0.75}, with the following equation (1-9). The inventors of the present invention use the term “TTOR” to mean a turnover rate of a ruminal liquid fraction per unit metabolic body weight per day. Note that the TTOR can be a turnover rate of a rumen liquid fraction per unit of values of length, weight, capacity, and the like related to a cow body and bovine excretions, such as a body weight, a weight of a rumen content derived from the body weight, a capacity of a rumen derived from the body weight, a blood volume derived from the body weight, an organ weight derived from the body weight, a body height, a fecal volume, a urine volume, and an expiratory volume.

TTOR((L/day)/MBW)=PRV/MBW (1-9):

(1-5. Measurement Result)

Parameters related to a DMI, a body weight, rumen fermentation, blood components, and milk components are listed in Table 1.

TABLE 1
Weeks after parturition

| | −3 | | 4 | | 8 | | 12 | |
|---|---|---|---|---|---|---|---|---|
| | | Standard | | Standard | | Standard | | Standard |
| Parameters | Average | deviation | Average | deviation | Average | deviation | Average | deviation |
| DMI (kg) | 12.3 | 1.3 | 21.8 | 3.0 | 24.5 | 2.6 | 25.7 | 2.8 |
| Body weight (kg) | 671.4 | 44.8 | 599.3 | 52.6 | 608.2 | 59.0 | 632.1 | 41.1 |
| Metabolic body weight | 131.8 | 6.5 | 121.0 | 8.0 | 122.4 | 8.9 | 126.0 | 6.1 |
| (body weight^{0.75}) | | | | | | | | |
| Rumen | | | | | | | | |
| pH | 6.72 | 0.15 | 6.55 | 0.48 | 6.38 | 0.81 | 6.78 | 0.31 |
| SCFA (mM) | 89.8 | 37.0 | 126.7 | 54.4 | 108.5 | 50.0 | 119.2 | 46.8 |
| Acetic acid (%) | 73.7 | 4.0 | 64.4 | 3.1 | 65.9 | 2.6 | 65.1 | 3.3 |
| Propionic acid (%) | 16.6 | 1.0 | 23.6 | 2.8 | 22.9 | 2.3 | 23.9 | 4.0 |
| Butyric acid (%) | 9.7 | 3.6 | 12.0 | 2.3 | 11.2 | 1.9 | 11.0 | 2.6 |
| Acetic acid/propionic | 4.48 | 0.48 | 2.78 | 0.51 | 2.91 | 0.35 | 2.80 | 0.56 |
| acid ratio | | | | | | | | |
| Lipopolysaccharide | 3882 | 1335 | 20496 | 10115 | 20254 | 7932 | 43704 | 27069 |
| (EU/ml) | | | | | | | | |
| Blood plasma | | | | | | | | |
| Total protein (g/dl) | 7.15 | 0.60 | 8.09 | 0.92 | 7.96 | 1.04 | 8.05 | 1.07 |
| Albumin (g/dl) | 3.65 | 0.22 | 3.78 | 0.46 | 3.60 | 0.42 | 3.74 | 0.34 |
| Globulin (g/dl) | 3.50 | 0.55 | 4.31 | 1.15 | 4.36 | 1.05 | 4.31 | 1.08 |
| Albumin/globulin ratio | 1.07 | 0.18 | 0.94 | 0.27 | 0.87 | 0.23 | 0.92 | 0.24 |
| Glucose (mg/dl) | 65.8 | 3.1 | 60.6 | 5.1 | 60.5 | 6.9 | 62.3 | 9.0 |
| Non-esterifed fatty acid | 0.188 | 0.258 | 0.164 | 0.113 | 0.106 | 0.069 | 0.076 | 0.028 |
| (mEq/l) | | | | | | | | |
| Triglyceride (mg/dl) | 16.1 | 7.5 | 6.1 | 1.8 | 6.1 | 1.6 | 87 | 5.6 |
| Total cholesterol (mg/dl) | 93.5 | 19.8 | 161.5 | 37.9 | 183.2 | 56.1 | 194.1 | 57.9 |
| AST (U/I) | 55.2 | 10.6 | 89.8 | 33.0 | 91.5 | 34.3 | 100.9 | 46.6 |
| GGT (U/I) | 20.1 | 4.7 | 37.9 | 29.4 | 33.5 | 16.0 | 31.8 | 15.1 |
| Blood urea nitrogen | 15.0 | 2.9 | 16.2 | 4.5 | 18.8 | 4.9 | 17.0 | 5.0 |
| Ca (mg/dl) | 9.24 | 0.68 | 9.63 | 0.70 | 9.32 | 0.85 | 9.32 | 0.77 |
| iP (mg/dl) | 5.74 | 1.39 | 5.01 | 1.12 | 4.97 | 1.12 | 5.28 | 1.24 |
| Milk | | | | | | | | |
| Milk yield (kg) | — | — | 42.7 | 7.1 | 45.8 | 5.8 | 43.5 | 6.0 |
| Milk yield/DMI | — | — | 2.0 | 0.4 | 1.9 | 0.3 | 1.7 | 0.3 |
| Milk fat (%) | — | — | 3.25 | 0.36 | 2.94 | 0.42 | 2.37 | 0.65 |
| Milk protein (%) | — | — | 2.96 | 0.17 | 2.95 | 0.27 | 2.99 | 0.23 |
| Solid non fat (%) | — | — | 8.34 | 0.18 | 8.44 | 0.22 | 8.48 | 0.21 |
| Ammonia nitrogen | — | — | 11.1 | 3.6 | 14.7 | 3.1 | 12.5 | 2.6 |
| (mg/dl) | | | | | | | | |

(1-6. Calculation of TTOR)

Based on the measurement results shown in Table 1, various parameters representing properties of rumen fermentation were calculated using the above equations (1-1) through (1-9), and are shown in Table 2.

TABLE 2
Weeks after parturition

| | −3 | | 4 | | 8 | | 12 | |
|---|---|---|---|---|---|---|---|---|
| | | Standard | | Standard | | Standard | | Standard |
| Parameters | Average | deviation | Average | deviation | Average | deviation | Average | deviation |
| HP (mM) | 182.5 | 76.5 | 254.8 | 111.4 | 217.5 | 101.2 | 236.9 | 95.6 |
| HUS (mM) | 47.5 | 21.6 | 91.3 | 43.0 | 73.9 | 33.5 | 81.5 | 28.6 |
| RM (mM) | 29.2 | 12.0 | 34.5 | 14.6 | 30.5 | 14.5 | 32.9 | 14.9 |
| Methane yield | 14.9 | 1.5 | 26.1 | 3.6 | 29.4 | 3.2 | 30.9 | 3.4 |
| (mol/day) | | | | | | | | |
| PRV (l/day) | 579.3 | 207.2 | 929.5 | 461.9 | 1197.9 | 598.7 | 1145.6 | 556.7 |
| SCFA yield | 45.8 | 4.7 | 96.0 | 13.0 | 105.6 | 11.6 | 115.3 | 19.7 |
| (mol/day) | | | | | | | | |
| SCFA yield/DMI | 3.71 | 0.09 | 4.42 | 0.31 | 4.32 | 0.27 | 4.47 | 0.50 |
| (mol/kg) | | | | | | | | |
| TTOR | 4.41 | 1.62 | 7.78 | 4.08 | 10.09 | 5.76 | 9.24 | 4.88 |

[2. Calculation of TTOR Based on Milk Yield and Milk Component]

Pieces of data below were calculated based on pieces of measured data (a body weight, a dry matter intake (DMI), concentrations of acetic acid, propionic acid, and butyric acid in a rumen liquid fraction, ruminal pH, a milk yield, and a milk component) disclosed in Reference Documents 2 through 8. Here, Reference Documents 2 through 8 are entirely incorporated as reference into the present specification.
Reference Document 2: Khafipour, E., Krause, D. O., & Plaizier, J. C. (2009). Alfalfa pellet-induced subacute ruminal acidosis in dairy cows increases bacterial endotoxin in the rumen without causing inflammation. Journal of Dairy Science, 92(4), 1712-1724.
Reference Document 3: Gao, X., & Oba, M. (2016). Characteristics of dairy cows with a greater or lower risk of subacute ruminal acidosis: Volatile fatty acid absorption, rumen digestion, and expression of genes in rumen epithelial cells. Journal of dairy science, 99(11), 8733-8745.
Reference Document 4: Hagg, F. M., Erasmus, L. J., Henning, P. H., & Coertze, R. J. (2010). The effect of a direct fed microbial (Megasphaera elsdenii) on the productivity and health of Holstein cows. South African Journal of Animal Science, 40(2) 101-112.
Reference Document 5: Tager, L. R., & Krause, K. M. (2011). Effects of essential oils on rumen fermentation, milk production, and feeding behavior in lactating dairy cows. Journal of dairy science, 94(5), 2455-2464.
Reference Document 6: Nasrollahi, S. M., Zali, A., Ghorbani, G. R., Shahrbabak, M. M., & Abadi, M. H. S. (2017). Variability in susceptibility to acidosis among high producing mid-lactation dairy cows is associated with rumen pH, fermentation, feed intake, sorting activity, and milk fat percentage. Animal feed science and technology, 228, 72-82.
Reference Document 7: Colman, E., Fokkink, W. B., Craninx, M., Newbold, J. R., De Baets, B., & Fievez, V. (2010). Effect of induction of subacute ruminal acidosis on milk fat profile and rumen parameters. Journal of dairy science, 93(10), 4759-4773.
Reference Document 8: Macmillan, K., Gao, X., & Oba, M. (2017). Increased feeding frequency increased milk fat yield and may reduce the severity of subacute ruminal acidosis in higher-risk cows. Journal of dairy science, 100(2), 1045-1054.

An estimation equation for estimating a TTOR from a milk yield and a milk component was obtained. As the estimation equation for estimating a TTOR from a milk yield and a milk component, a plurality of equations can be prepared. Here, as an example, an estimation equation for estimating a TTOR from a total milk protein amount was obtained. The total milk protein amount can be calculated from a milk yield and a milk protein concentration that are readily measurable even by an ordinary farmer. First, the total milk protein amount was obtained from a milk yield and a milk protein concentration disclosed in Reference Documents 2 through 8.

Milk total protein (MTP)(g/day)=milk yield(g/day)×milk protein concentration(%)

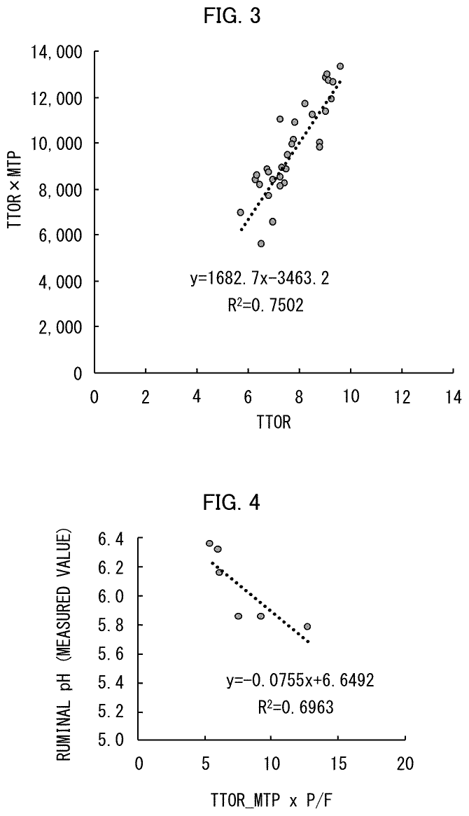

Next, with use of a TTOR calculated from pieces of measured data disclosed in Reference Documents 2 through 8, a product (TTOR×MTP) of the TTOR and the MTP was obtained in a manner similar to that described in 1. above. A scatter diagram between this TTOR×MTP and the TTOR is illustrated in FIG. 3.

In the scatter diagram of FIG. 3, a regression line was obtained by the least squares method. From the obtained regression line, the following equation is obtained, where “TTOR_MTP” represents a TTOR estimated from the MTP, x=TTOR_MTP. and z=MTP.

xz=1682.7×−3463.2

Therefore, x=2948.1/(1597.7−z).

That is, the TTOR_MTP can be calculated from an equation (2-1): TTOR_MTP=2948.1/(1597.7−MTP).

Here, an average value of TTOR_MTP calculated from the equation (2-1) was 8.48, which was higher than an average value 7.74 of the TTOR calculated in 1-6 above, but no significant difference was observed in a t-test.

That is, it was indicated that a TTOR can be estimated from a milk yield and a milk component without measuring a DMI and rumen fluid components.

[3. Estimation of Ruminal pH from TTOR Based on Milk Yield and Milk Component]

An estimation equation for estimating ruminal pH from a milk yield, a milk component, and TTOR_MTP was obtained. As the estimation equation for estimating ruminal pH from a milk yield, a milk component, and TTOR_MTP, a plurality of equations can be prepared. Here, as an example, an estimation equation for estimating ruminal pH from TTOR_MTP and a ratio (P/F) between a milk protein proportion (P) and a milk fat proportion (F) was obtained.

First, TTOR_MTP×P/F was calculated based on pieces of measurement data described in Reference Documents 2 through 8.

Then, a correlation between the calculated value of TTOR_MTP×P/F and measured values of ruminal pH measured in Reference Documents 2 through 8 was obtained by regression analysis. The following estimation equation (3-1) was obtained under conditions in which TTOR_MTP is 6 or more and TTOR_MTP×P/F is less than 17.

Ruminal pH=−0.0239×(TTOR_MTP×P/F)+6.1824 (3-1):

When estimated ruminal pH was calculated by substituting TTOR_MTP×P/F into the equation (3-1), an average of the estimated ruminal pH was 6.00, which was close to an average 5.96 of a measured value of ruminal pH. No significant difference was observed in the t-test.

However, a multiple correlation coefficient (R²) of the equation (3-1) was low, i.e., 0.107, and it was thus suggested that some pieces of data show a large difference between the estimated ruminal pH and the measured value of ruminal pH.

As a method for correcting this drawback, the following study was carried out. The equation (3-1) is an estimation equation in which the pieces of measurement data disclosed in Reference Documents 2 through 8 are used as samples. Study was carried out in which the pieces of measurement data disclosed in Reference Documents were used as respective samples, and estimation equations corresponding to the equation (3-1) were obtained for the respective samples to calculate estimated ruminal pH more accurately. In Reference Documents 2 through 8, pieces of data are measured for dairy cows of different rearing conditions at different institutes (farms). Therefore, obtaining an estimation equation corresponding to the equation (3-1) for each of Reference Documents means to obtain an estimation equation for each farm.

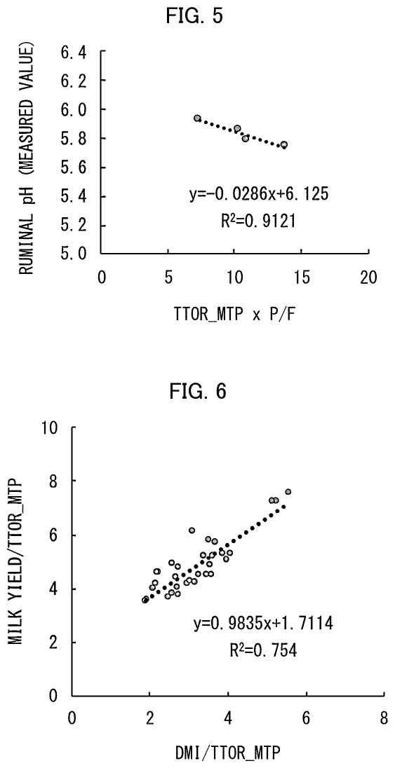

As an example, scatter diagrams between (i) TTOR_MTP×P/F calculated using the measured data disclosed in Reference Documents 2 and 4 as samples and (ii) measured values of ruminal pH measured in Reference Documents 2 and 4 are illustrated in FIG. 4 and FIG. 5, respectively. FIG. 4 is a scatter diagram using the measurement data disclosed in Reference Document 2 as a sample, and FIG. 5 is a scatter diagram using the measurement data disclosed in Reference Document 4 as a sample. On the basis of the scatter diagrams of FIGS. 4 and 5, regression lines were obtained by the least squares method, respectively, and regression equations were obtained, respectively.

Estimated ruminal pH was calculated by substituting TTOR_MTP×P/F calculated based on the measured data described in each of Reference Documents 2 and 4 into the equation obtained from the measured data described in each of Reference Documents 2 and 4. As a result, an average of the estimated ruminal pH was 5.96 (standard deviation: 0.20), and an average of measured values of ruminal pH was an extremely close value, i.e., 5.96 (standard deviation: 0.22).

Estimated ruminal pH based on the measurement data disclosed in Reference Documents 2 and 4 and measured values of ruminal pH disclosed in Reference Documents 2 and 4 are indicated in Table 3 below. In Table 3, the estimated ruminal pH obtained from the equation (3-1) was referred to as “estimated ruminal pH (A)”. In addition, in Table 3, the estimated ruminal pH obtained from the equation obtained from the measurement data disclosed in each of Reference Documents 2 and 4 was referred to as “estimated ruminal pH (B)”.

TABLE 3

| | Reference Document 2 | | |
|---|---|---|---|
| | Estimated | Estimated | |
| | ruminal | ruminal | Ruminal pH |
| | pH (A) | pH (B) | (measured value) |
| | 6.05 | 6.23 | 6.35 |
| | 6.04 | 6.19 | 6.31 |
| | 6.03 | 6.18 | 6.15 |
| | 6.00 | 6.07 | 5.85 |
| | 5.96 | 5.94 | 5.85 |
| | 5.87 | 5.68 | 5.78 |
| Average | 5.99 | 6.05 | 6.05 |
| Standard | 0.067 | 0.211 | 0.253 |
| deviation | | | |
| | Reference Document 4 | | |
| | Estimated | Estimated | Ruminal pH |
| | ruminal | ruminal | measured |
| | pH (A) | pH (B) | value J |
| | 5.94 | 5.83 | 5.85 |
| | 6.01 | 5.92 | 5.92 |
| | 5.92 | 5.81 | 5.78 |
| | 5.85 | 5.73 | 5.74 |
| Average | 5.93 | 5.82 | 5.82 |
| Standard | 0.132 | 0.132 | 0.079 |
| deviation | | | |

Thus, it has been indicated that ruminal pH which is a value close to a measured value of ruminal pH can be estimated by using an estimation equation obtained by regression analysis of samples from a plurality of farms or a sample from each farm. In other words, it has been indicated that ruminal pH can be estimated by obtaining an estimation equation of ruminal pH from a milk yield, a milk component and a TTOR without collecting rumen fluid, and in particular, ruminal pH can be estimated more accurately by obtaining an estimation equation of ruminal pH for each farm. As shown in 1. above, it is also useful to use an estimation equation for pH obtained from samples of a data group obtained at different times for each cow to analyze the individual cow.

[4. Estimation of DMI from TTOR Based on Milk Yield and Milk Component]

An estimation equation for determining a DMI from a milk yield, a milk component, and TTOR_MTP was obtained. Here, as an example, an estimation equation for estimating a DMI from a milk yield and TTOR_MTP was obtained.

First, milk yield/TTOR_MTP and DMI/TTOR_MTP were calculated based on the pieces of measurement data disclosed in Reference Documents 2 through 8. A scatter diagram between the calculated milk yield/TTOR_MTP and DMI/TTOR_MTP is illustrated in FIG. 6.

In the scatter diagram of FIG. 6, a regression line was obtained by the least squares method. From the obtained regression line, the following equation is obtained, where “DMI_(TTOR_MTP)” represents an estimated DMI estimated from TTOR_MTP, x=DMI_(TTOR_MTP), z=TTOR_MTP, and milk yield=MY.

MY/z=0.9835x(x/z)+1.7114

Therefore, x=(MY−1.7114z)/0.9835.

That is, DMI_(TTOR_MTP) can be calculated from an equation (4-1): DMI_(TTOR_MTP)=(MY−1.7114×TTOR_MTP)/0.9835.

When the estimated DMI (DMI_(TTOR_MTP)) was calculated by substituting the milk yield and TTOR_MTP into the equation (4-1), an average of the estimated DMI was 24.05 kg, which was close to an average 24.13 kg of measured values of DMI. No significant difference was observed in the t-test. A total of estimated DMIs (31 DMIs) was 745.6 kg, and a total of measured values of DMI (31 DMIs) was 747.9 kg. Therefore, when the data used is regarded as one cow group, the calculated total of estimated DMIs is close to the total of measured values of DMI, and this is very useful in determining an amount of feed to be supplied.

However, a multiple correlation coefficient (R²) of the equation (4-1) was low, i.e., 0.0041, and it was thus suggested that some pieces of data show a large difference between the estimated DMI and the measured value of DMI.

As a method for correcting this drawback, the following study was carried out. The equation (4-1) is an estimation equation in which the pieces of measurement data disclosed in Reference Documents 2 through 8 are used as samples. Study was carried out in which the pieces of measurement data disclosed in Reference Documents were used as respective samples, and estimation equations corresponding to the equation (4-1) were obtained for the respective samples to calculate estimated DMIs more accurately. In Reference Documents 2 through 8, pieces of data are measured for dairy cows of different rearing conditions at different institutes (farms). Therefore, obtaining an estimation equation corresponding to the equation (4-1) for each of Reference Documents means to obtain an estimation equation for each farm.

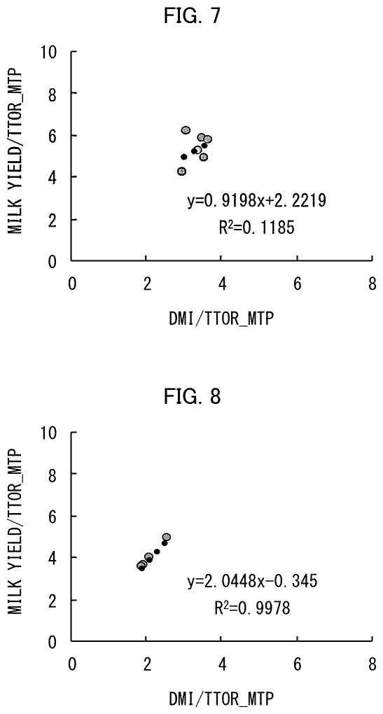

As an example, milk yield/TTOR_MTP calculated using the pieces of measurement data disclosed in Reference Documents 2 and 4 as samples and DMI/TTOR_MTP were calculated. A scatter diagram between the calculated milk yield/TTOR_MTP and DMI/TTOR_MTP is illustrated in each of FIGS. 7 and 8. FIG. 7 is a scatter diagram using the measurement data disclosed in Reference Document 2 as a sample, and FIG. 8 is a scatter diagram using the measurement data disclosed in Reference Document 4 as a sample. On the basis of the scatter diagrams of FIGS. 7 and 8, regression lines were obtained by the least squares method, respectively, and regression equations were obtained, respectively.

An estimated DMI was calculated by substituting a milk yield and TTOR_MTP into the obtained regression equation. Estimated DMIs based on the measurement data disclosed in Reference Documents 2 and 4 and measured values of DMI disclosed in Reference Documents 2 and 4 are indicated in Table 4 below.

TABLE 4
Reference Document 2

| | Estimated | Estimated | |
|---|---|---|---|
| | DMI | DMI | DMI |
| | (A) | (B) | (measured value) |
| | 26.3 | 24.9 | 18.1 |
| | 25.6 | 23.9 | 21.6 |
| | 24.2 | 22.5 | 21.7 |
| | 23.1 | 21.1 | 22.0 |
| | 21.2 | 19.0 | 23.4 |
| | 19.6 | 16.5 | 23.4 |
| Average | 23.3 | 21.3 | 21.7 |
| Standard | 2.58 | 3.13 | 1.94 |
| deviation | | | |

Reference Document 4

| | Estimated | Estimated | |
|---|---|---|---|
| | DMI | DMI | DMI |
| | (A) | (B) | (measured value) |
| | 25.4 | 23.3 | 23.1 |
| | 28.3 | 22.4 | 22.4 |
| | 24.0 | 24.2 | 24.3 |
| | 23.2 | 23.7 | 23.8 |
| Average | 25.2 | 23.4 | 23.4 |
| Standard | 2.22 | 0.78 | 0.83 |
| deviation | | | |

As such, the value close to the DMI (measured value) can be obtained by the manner of calculating an estimated DMI (estimated DMI (B)) using the regression equation for each farm.

Thus, it has been indicated that a DMI which is a value close to a measured value of DMI can be estimated by using an estimation equation obtained by regression analysis of samples from a plurality of farms or a sample from each farm. In other words, it has been indicated that a DMI can be estimated by obtaining an estimation equation of DMI from a milk yield, a milk component and a TTOR without weighing supplied feed and residual feed, and in particular, a DMI can be estimated more accurately by obtaining an estimation equation of DMI for each farm. As shown in 1. above, it is also useful to use an estimation equation for DMI obtained from samples of a data group obtained at different times for each cow to analyze the individual cow.

### INDUSTRIAL APPLICABILITY

The present invention can be utilized in the agricultural field, particularly with respect to dairy farming.

### REFERENCE SIGNS LIST

10: Measurement device
20: Evaluation device
21: Estimation unit
22: Management information generation unit
23: Diagnosis unit
24: Storage unit
25: Calculation unit
100: Evaluation system

1. An evaluation system for evaluating a status of rumen fermentation in a ruminant, said evaluation system comprising:
an evaluation device that has an estimation unit, the estimation unit estimating a status of rumen fermentation in an evaluation subject ruminant based on a milk production record which indicates a milk yield and a milk component of milk produced by the evaluation subject ruminant.

2. The evaluation system as set forth in claim 1, wherein:
the estimation unit estimates a theoretical turnover rate of a rumen liquid fraction based on the milk production record of milk produced by the evaluation subject ruminant, the theoretical turnover rate indicating the status of rumen fermentation in the evaluation subject ruminant.

3. The evaluation system as set forth in claim 2, wherein:
the estimation unit estimates the theoretical turnover rate of the rumen liquid fraction of the evaluation subject ruminant from a measurement result in the milk production record of the evaluation subject ruminant based on a correlation between (i) a theoretical turnover rate of a rumen liquid fraction which has been calculated based on a methane yield released by a ruminant and a ruminal short-chain fatty acid concentration in the ruminant and (ii) the milk production record.

4. The evaluation system as set forth in claim 2, wherein:
the estimation unit estimates ruminal pH of the evaluation subject ruminant from a measurement result in the milk production record of the evaluation subject ruminant based on an estimated ruminal theoretical turnover rate which has been estimated from the milk production record.

5. The evaluation system as set forth in claim 2, wherein:
the estimation unit estimates a dry matter intake from a measurement result in the milk production record of the evaluation subject ruminant based on an estimated ruminal theoretical turnover rate which has been estimated from the milk production record.

6. The evaluation system as set forth in claim 1, wherein:
the evaluation device further includes a management information generation unit that generates feeding management information pertaining to a ruminant based on a status of rumen fermentation in the ruminant, which has been estimated by the estimation unit.

7. The evaluation system as set forth in claim 1, wherein:
the evaluation device further includes a diagnosis unit that diagnoses at least one of a feeding management status and a production disease of a ruminant based on a status of rumen fermentation in the ruminant, which has been estimated by the estimation unit.

8. The evaluation system as set forth in claim 1, wherein:
the evaluation device further includes a storage unit that stores information indicating a correlation between the milk production record and the status of rumen fermentation in the ruminant.

9. The evaluation system as set forth in claim 1, wherein:
the evaluation device further includes a calculation unit that calculates a relational expression indicating a correlation between the milk production record and the status of rumen fermentation in the ruminant.

10. The evaluation system as set forth in claim 1, wherein:
the milk component includes at least one of a milk fat proportion, a milk protein proportion, a solid non fat proportion, a milk sugar proportion, an ammonia nitrogen proportion, and a milk protein/milk fat ratio.

11. The evaluation system as set forth in claim 10, wherein:
the milk production record includes at least one value calculated from a combination of two or more values of the milk component.

12. An evaluation method for evaluating a status of rumen fermentation in a ruminant, said method comprising:
an estimation step of estimating a status of rumen fermentation in a ruminant based on a milk production record which indicates a milk yield and a milk component of milk produced by the ruminant.
